(19) United States
(12) Patent Application Publication
(10) Pub. No.: US 2010/0261790 A1
(43) Pub. Date: Oct. 14, 2010

(54) BODY-WEIGHT MAINTENANCE AND BODY COMPOSITION

(76) Inventors: Bengt Herslöf, Stockholm (SE); Annika Viberg, Jarfalla (SE)

Correspondence Address: NIXON & VANDERHYE, PC, 901 NORTH GLEBE ROAD, 11TH FLOOR, ARLINGTON, VA, 22203, US

(21) Appl. No.: 12/598,787

(22) PCT Filed: May 8, 2008
(86) PCT No.: PCT/EP08/55713

§ 371 Date: May 6, 2010

(30) Foreign Application Priority Data
May 8, 2007 (EP) 07107719.2

(51) Int. Cl. A61K 31/23; A61P 3/00; A61P 9/00

(52) U.S. Cl. 514/547

(57) ABSTRACT

Use of a mixture comprising a triglyceride oil having a solid fat content at ambient to body temperature and an emulsifier for the preparation of a pharmacologically active composition for increasing body fat mass loss or maintaining blood pressure in an individual. Such use may be carried out subsequent to a period of weight loss. Preferably, the triglyceride oil is fractiorated oil and the emulsifier is galactolipid.

### FIELD OF THE INVENTION

This invention is in the field of weight maintenance, more in particularly in the field of long term weight maintenance of humans. Accordingly, the invention relates to increasing body fat mass loss and to maintaining blood pressure.

### BACKGROUND TO THE INVENTION

The increasing incidence of obesity is a recognized medical problem in developed countries. Obesity is a major factor for a number of diseases, including coronary heart diseases, hypertension, non-insulin dependent diabetes mellitus, pulmonary dysfunction, osteoarthritis and certain types of cancer. Obesity develops when the equilibrium between energy intake and energy expenditure shifts towards a positive energy balance.

Obesity can be classified as a mild (20-30% overweight), moderate (30-60% overweight) or a severe (>60% overweight) condition. Obesity is accompanied by a number of health hazards. It may impair both cardiac and pulmonary functions, perturb endocrine functions and cause emotional problems. Hypertension, impaired glucose tolerance and non-insulin dependant diabetes mellitus and hypercholesterolemia are more common conditions in overweight individuals than in individuals of normal weight. Obesity may therefore contribute to morbidity and mortality in individuals suffering from e.g. hypertension, stroke, diabetes mellitus type II, some types of cancer, gallbladder disease and ischaemic heart disease. Moderate and severe cases of obesity are known to increase mortality. Colon and rectal cancer are diseases which frequently appear in obese men, and obese women often suffer from endometrium or gallbladder cancer. Furthermore, it is realized that an increase in overweight almost consequently leads to a rise in psychological and social problems.

Treatment of obesity is beneficial in that weight loss reduces the risk for mortality and morbidity. Even modest weight loss, for example 5 to 10% of the initial body-weight is from a medical point of view interesting. A weight reduction may be attained by increased energy consumption and/or decreased intake of energy. After weight reduction is attained, it is important not to return to the earlier food intake. The risk with fast weight reduction, without subsequently maintaining the reduced weight, is of developing so called “yo-yo” dieting, with further increase in weight as a consequence.

### SUMMARY OF THE INVENTION

The inventors have studied the effects of a mixture of a triglyceride oil, which triglyceride oil optionally has a solid fat content at ambient to body temperature and an emulsifier, preferably a food emulsifier, on individuals subject to a negative energy balance.

Individuals subject to a meal replacement regime in which the mixture is consumed lose body fat mass to a significantly higher degree than individuals subject to the same meal replacement regime without the mixture, but with the same fat content.

During the weight loss phase, systolic blood pressure decreases. After the weight loss phase, individuals subject to a meal replacement regime in which the mixture is consumed maintain systolic blood pressure, whereas blood pressure increases significantly in individuals subject to the same meal replacement regime without the mixture, but with the same fat content.

According to the invention, there is thus provided use of a mixture of a triglyceride oil and an emulsifier for increasing body fat mass loss in an individual.

The invention also provides use of a mixture of a triglyceride oil and an emulsifier for the preparation of a pharmacologically active composition, i.e. medicament, for increasing body fat mass loss in an individual.

According to the invention, there is further provided use of a mixture of a triglyceride oil and an emulsifier for maintaining blood pressure in an individual.

The invention also provides use of a mixture of a triglyceride oil and an emulsifier for the preparation of a pharmacologically active composition, i.e. medicament, for maintaining blood pressure in an individual.

The triglyceride oil may be one which has a solid fat content at ambient to body temperature

In the uses described herein, it will be evident to the skilled person that the mixtures may comprise components other than a triglyceride oil having a solid fat content at ambient to body temperature and an emulsifier. That is to say, the uses described herein concern uses of mixtures comprising a triglyceride oil, optionally having a solid fat content at ambient to body temperature, and an emulsifier.

The invention also provides:

a mixture of a triglyceride oil, optionally having a solid fat content at ambient to body temperature, and an emulsifier for the preparation of a pharmacologically active composition (medicament) for increasing body fat mass loss in an individual;

a mixture of a triglyceride oil, optionally having a solid fat content at ambient to body temperature, and an emulsifier for the preparation of a pharmacologically active composition (medicament) for maintaining blood pressure in an individual;

a method for increasing body fat mass loss in an individual, which method comprises the step of administering to the individual a therapeutically effective amount of a mixture of a triglyceride oil, optionally having a solid fat content at ambient to body temperature, and an emulsifier; and

a method for maintaining blood pressure in an individual, which method comprises the step of administering to the individual a therapeutically effective amount of a mixture of a triglyceride oil, optionally having a solid fat content at ambient to body temperature, and an emulsifier.

The invention also relates to the use of a mixture essentially consisting of a triglyceride oil, optionally having a solid fat content at ambient to body temperature, and an emulsifier for the preparation of a pharmacologically active composition, i.e. medicament, for increasing body fat mass loss or for maintaining blood pressure. Such a mixture may of course additionally comprise colorants, antioxidants etc. Such a mixture will typically be in the form of an oil-in-water emulsion.

Such use is particularly advantageous for maintaining weight after a period of weight loss and/or when the individual is subject to a negative energy balance, for example when subject to a meal replacement regime.

The invention further provides a product comprising:

a mixture comprising a triglyceride oil, optionally having a solid fat content at ambient to body temperature, and an emulsifier; and

a meal replacement product.

Such a product may be used for increasing body fat mass loss in an individual and/or for maintaining blood pressure in an individual.

### BRIEF DESCRIPTION OF THE DRAWINGS

FIG. 1 shows a schematic representation of the study design: V1 to V7=visit 1 to visit 7.

FIG. 2 shows a schematic view of how the waist and hip measurements were performed.

FIG. 3 shows a box plot for body fat mass in % for active and placebo treatments.

FIG. 4 shows a box plot for body muscle mass in % for active and placebo treatments.

FIG. 5 shows the body fat mass for the active and placebo treatments plotted against time.

### DETAILED DESCRIPTION OF THE INVENTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

This invention relates to the use of a mixture in order to increase body fat mass loss and/or to maintain blood pressure. The mixture may be used in this way for a health, for example therapeutic, reason (for example in an individual who is classified as overweight), for a prophylactic reason or for a cosmetic or other non-therapeutic reason (for example in an individual who is not classified as being overweight).

The mixture comprises a triglyceride oil and an emulsifier. The triglyceride oil may have a solid fat content at ambient to body temperature The mixture may consist essentially of a triglyceride oil, optionally having a solid fat content at ambient to body temperature, and an emulsifier. The mixtures/compositions that may be used in the invention are described in more detail below.

When the mixtures, or compositions, described herein were used as a food supplement for human individuals after a period of weight loss and in the context of a dietary regime conferring a negative energy balance, it was observed that body fat mass loss in the individuals receiving a composition according to the invention (test individuals) was significantly higher than in those individuals that did not receive any of the compositions (control individuals). Also, the blood pressure reduction observed during the weight loss period was maintained in the test individuals, whereas blood pressure increased in the control individuals.

In more detail, after a weight loss phase, the participants in the study reported in the Examples continued to replace one meal (lunch) with a meal replacement product containing Fabuless™ (DSM Food Specialties, Delft, The Netherlands) or cream (Low Calorie Diet). On this negative energy balance during the intervention phase, the subjects continued to lose body fat mass, and to a significantly higher degree, in the Fabuless™ group (a statistically significant difference, p=0.023, could be observed after 8 and 12 weeks consumption).

The systolic blood pressure was decreased as expected during the weight loss period. Despite the same amount of fat being present in the two test products used during the intervention, the systolic blood pressure was maintained in the Fabuless™ group, but significantly increased from baseline in the placebo group.

The term “increase/increased/increasing body fat mass loss”, and the like, implies that the body fat mass loss in an individual consuming the mixture described herein is greater than the body fat mass loss in an individual subject to substantially the same dietary regime, for example wherein the mixture were to be replaced with an equivalent amount of fat (such as fat from milk). That is to say, an individual consuming the mixture described herein will lose body fat mass to a greater extent than an individual subject to a calorifically similar (or identical) dietary regime in which the mixture is not consumed.

Accordingly, the body fat mass loss will be greater in an individual consuming the mixture described herein than in an individual which does not consume the mixture. The body fat mass loss in an individual consuming the mixture may be at least about 10%, at least about 50%, at least about 100% or at least about 500% greater than the body fat mass loss in an individual not consuming the said mixture. Such differences may be observed a period of about 4 weeks, about 8 weeks, about 12 weeks or longer after the individual has commenced taking the mixture.

The term “maintain blood pressure” implies that blood pressure, for example systolic blood pressure, in an individual consuming the mixture described herein rises less than in an individual subject to substantially the same dietary regime, for example wherein the mixture were to be replaced with an equivalent amount of fat (such as fat from milk).

Typically, “maintain” in this context implies that the blood pressure does not rise more than about 50%, more than about 20%, more than about 10% or more than about 5%, more than about 10%, more than about 5% the rise in blood pressure that would be seen in an individual subject to substantially the same dietary regime, for example wherein the mixture were to be replaced with an equivalent amount of fat (such as fat from milk).

“Maintain” may thus be defined as an increase in blood pressure of no more than about 20%, for example no more than about 10%, such as no more than 5% of the initial blood pressure of an individual (prior to consumption of the mixture described herein, for example after a period of weight loss). “Maintain” herein may imply that blood pressure does not rise to an extent which is statistically significant.

In this context, maintaining blood pressure may refer to keeping an approximate blood pressure loss which was achieved during the period of weight loss. Blood pressure may be considered maintained when blood pressure regain, 4 weeks, 8 weeks, 12 weeks or longer after the end of the period of blood pressure loss, as a % of blood pressure loss, does not exceed about 35%, such as about 30%, about 25%, about 20% or even about 15% or less.

The mixture described herein may be used after a period of weight loss. Such weight loss may have been accomplished by treatment intervention or by an individual's own efforts. Typically, the term “weight loss” refers to achieving a weight loss of at least about 2% of initial or baseline body weight, such as at least about 3%, at least about 4%, at least about 5%, at least about 7%, at least about 10% or even at least about 15%. Such weight loss may be achieved over a period of from, for example, one, two or three to five, six, ten, eighteen or more weeks. Alternatively, weight loss may also be expressed as losing 2 or more body mass index (BMI) points over the period set out above.

Body fat mass (or body fat percentage) is the fraction of total body mass that is adipose tissue, as opposed to lean body mass (muscle, bone, organ tissue, blood, and everything else). Total body fat mass consists of fat and storage fat. Essential fat is that amount necessary for maintenance of life and reproductive functions. Storage fat consists of fat accumulation in adipose tissue, part of which protects internal organs in the chest and abdomen.

The body fat mass of an individual cannot generally be determined exactly. However, it may be estimated in a number of different ways. Any method known to those skilled in the art may be used to measure body fat mass for the purposes of this invention. Such methods include: dual energy X-ray absorptiometry (DXA); body average density measurement, for example using the Brozek [BF=(4.57/ρ-4.142)×100] or Siri [BF=(4.95/ρ-4.50)×100] formulae, in which body density is determined by, for example, hydrostatic weighing; bioelectrical impedance analysis; or anthropometric methods (using measurements of various parameters of the human body, such as circumferences of various body parts or thicknesses of skinfolds, for example using formulae such as the Durnin-Womersley skinfold method (Durnin and Womersley, Br. J. Nutr. (1974), 32, 77), the Jackson-Pollock skinfold method or the US Navy circumference method which estimate body density which may then be converted to body fat mass using formulae such as the Brozek or Siri formulae).

Blood pressure refers to the force exerted by circulating blood on the walls of blood vessels. Blood pressure herein typically refers to arterial blood pressure, i.e. the pressure in the larger arteries. Generally, blood pressure herein may refer to systolic pressure, i,e, the peak pressure in the arteries, which occurs near the beginning of the cardiac cycle. Blood pressure herein may though refer to an average pressure throughout the cardiac cycle, for example mean arterial pressure.

Blood pressure may be measured invasively (by penetrating the skin and measuring inside the blood vessels) or non-invasively.

Non-invasive methods include auscultatory methods and oscillometric methods. Auscultatory methods generally comprises the use of a sphygnanometer and a stethoscope. The sphygnanometer may comprise a mercury or an aneroid manometer. Oscillometric methods are functionally similar to auscultatory methods, but generally use an electronic pressure sensor (transducer) fitted in to detect blood flow, instead of using a stethoscope.

Invasive methods typically involve measurement with intravascular cannulas and direct measurement of arterial pressure. Invasive Blood Pressure Monitors are pressure monitoring systems designed to acquire pressure information for display and processing. A variety of invasive blood pressure monitors are available including single pressure, dual pressure, and multi-parameter. The monitors may be used for measurement and follow-up of, for example, arterial, central venous, pulmonary arterial, left atrial, right atrial pressures.

An individual using a mixture according to the invention will typically be subject to a dietary regime which results in a negative energy balance. Energy balance is defined as energy intake minus energy output. An individual is described as being in negative energy balance in the event that energy intake is insufficient to meet the requirements of maintenance and production. That is to say, an individual in negative energy balance is one wherein calorie intake (from food and drinks) is less than calorie expenditure (through metabolism and energy expended during daily activities).

An individual is in neutral energy balance when the energy intake is approximately equal to the energy output. For the purposes of this invention, an individual subject to a negative energy balance may be at any degree of negative energy balance. Typically, the individual will be in negative energy balance as determined on a daily basis, although an individual may be in negative energy balance for the purposes of this invention as determined over a period of time longer or shorter than one week, for example over a period of about 12 hours or over a period of about 1 week, about two weeks, about 6 weeks or longer, preferably for the entire period that the mixture described herein is consumed.

An individual subject to negative energy balance may be one in which the energy intake is about 90% or less, about 80% or less, about 70% or less, about 60% or less or about 50% or less than the energy intake required to achieve a neutral energy balance.

The calorie intake required to maintain a neutral energy balance will vary according to a wide number of variables. However, the recommend calorie intake for a woman leading typical moderately active lifestyle is in the region of from about 2000 to about 2200 kilocalories per day. The figure for a moderately active man is from about 2500 to about 2800 kilocalories per day. These figures may though need to be adjusted for age, for example old (over about 70 years old) or young (under about 10 years old) individuals generally require a lower energy intake to achieve a neutral energy balance. Also, very active individuals are likely to require a higher energy intake to achieve a neutral energy balance. A dietician will be able to advise as to the approximate energy intake required to achieve a neutral energy balance (and, therefore, the energy intake required to achieve a specific, desired degree of negative energy balance).

Accordingly, for the purposes of this invention, an individual may be generally considered in negative energy balance if they consume less than from about 2000 to about 2200 kilocalories per day (for a female) or less than from about 2500 to 2800 kilocalories per day (for a male). However, individuals consuming more than these energy amounts may, nevertheless, be in negative energy balance depending on their specific circumstances.

Such a dietary regime, where an individual is in negative energy balance, may be referred to as a Low Calorie Diet (LCD). A diet which achieves an energy intake of 800 kcal or less is defined as a Very Low Calorie Diet (VLCD). Individuals using a mixture according to the invention may be subject to a LCD or VLCD.

The Swedish Food Administration advises that three meals are taken each day along with 1 to 3 snacks in order to achieve neutral energy balance. The distribution of energy is recommended to be as follows: breakfast—from about 20% to about 25%; lunch—from about 25% to about 35%; and dinner—from about 25% to about 35%. This suggests that energy intake at lunch may be from about 500 kcal to about 770 kcal (women) and from about 625 kcal to about 890 kcal (men). Negative energy balance may thus be achieved by reducing the energy intake at one or more meals, for example lunch (with reference to the energy amounts set out above) and/or dinner and/or breakfast, whilst maintaining a normal (neutral) energy intake for the remaining meal or meals.

An individual using a mixture in accordance with the invention may be subject to a meal replacement regime. This may be a convenient way in which a negative energy balance may be achieved. In such regimes, meal replacements, for example in the form of a liquid or a solid bar, are consumed by the individual in place of one, two or more regular daily meals. In addition, the dieter may consume one, two or more meals of real food (which may be calorie-controlled, for example providing from about 400 kcal to about 600 kcal per day). Some liquid diet programs offer pre-packaged meal-options for these “real” meals. Meal replacement products contain typically from about 100 to about 400 kcalories, for example from about 150 to about 250 kcal. They may contain at least about 25% protein and at least about 3 vitamins and minerals. Most commercially-available products contain around 5 to 6 g fibre.

Such a diet may typically provide a total energy intake of from about 1000 kcal to about 1500 kcal per day, for example from about 1200 kcal to about 1400 kcal per day.

Accordingly, the invention provides a product comprising: a mixture comprising a triglyceride oil, optionally having a solid fat content at ambient to body temperature, and an emulsifier; and a meal replacement product.

In such a product, the mixture may be as defined herein. The meal replacement product may be as defined herein. The meal replacement product may be in any suitable form, such as a bar, a powder (for example, soup powder) or a liquid (such as a shake).

The product may provide multiple servings/dosages of the mixture and/or the meal replacement product. For example, the product may provide enough dosages for consumption over a period of one week, two weeks, three weeks, four weeks or more.

The meal replacement product may provide from about 100 to about 400 kcalories per serving, for example from about 150 to about 250 kcal.

Such a product may be a combined preparation, wherein the mixture and the meal replacement product are for simultaneous, separate or sequential use. Such a product may be for use in increasing body fat mass loss in an individual and/or in maintaining blood pressure in an individual.

An individual using a mixture according to the invention may be subject to a VLCD. This is defined medically as a diet of 800 kcal per day or less. VLCDs are formulated, nutritionally complete, liquid meals. VLCDs also contain the recommended daily requirements for vitamins, minerals, trace elements, fatty acids and protein. The VLCD products are usually a powder which is mixed with water, juice, or other low calorie liquid. Such diets are typically undertaken with medical supervision.

The mixtures that may be used in the invention are advantageously oil-in-water emulsions. In this application, the term “Oil-in-water emulsions” refers to liquid oil dispersions as well as to solid fat dispersions, that is suspensions. The amount of triglyceride oils (wt %) may vary depending on the envisaged application and the nature and characteristics of the triglyceride oil as is taught herein. It can be envisaged that a composition according to the invention contains 5, 10, 15, 20, 30, 40, or even 60 or more wt % of triglyceride oils up to maximum dispersability, i.e. when there is still a water continuous phase.

With the phrase “having a solid fat content at ambient to body temperature” it is meant that there should be a solid fat content in the whole interval between ambient and body temperature. The meaning of “a solid fat content” is known to the skilled person and may be determined using standard methodology, as for instance is provided at www.minispec.com/applications/solid fat content.html. Expressed in another way, the term means that there should be at least a residual and detectable solid fat content at body temperature. Residual and detectable solid fat contents may be in the order of more than 0.1%, such as 0.5%, 1%, 2%, 3%, 5%, 10% or more. Solid fat content may be determined by Benchtop NMR using ISO 8292 or IUPAC 2.150 methods. These methods yield a melting curve from which it can be easily determined whether a given triglyceride oil has a solid fat content in the range of ambient to body temperature. Additional methods for determining solid fat content include AOCS methods: AOCS Cd 16b-93 revised in 2000; Direct Method; and AOCS Cd 16-81 revised in 2000, Indirect Method.

Ambient temperature is used to indicate approximate room temperature being the temperature wherein the composition is used according to the invention. Usually this is approximately 20° C., such as 18, 19, 20, 21 or 22° C.

Body temperature differs slightly from species to species, herein this term is used to indicate the body temperature of the human individual to be treated. Usually this is approximately 37° C., such as 36, 36.5, 37, 37.5 38, 38.5 or 39° C.

The invention is particularly useful for increasing body fat mass loss in overweight or obese individuals. For practical purposes, it is generally agreed that overweight is present if the body weight exceeds the “desirable weight”, whereas obesity is present if the body weight is 20% or more above the “desirable weight”. Desirable weights for humans can be defined according to Metropolitan Height and Weight Tables as the midpoint of the range of the medium-frame individuals. The invention may though be useful in a non-overweight, non-obese individual who wishes to lose body fat mass for cosmetic purposes.

The term “Triglyceride” as used herein refers to triacylglycerol, that is glycerol esterified to three fatty acids.

The triglyceride oils of said mixtures or oil-in-water emulsions can be any triglyceride material, or triglyceride-containing material. A triglyceride oil for use in the invention may have a solid fat content at ambient to body temperature. The triglyceride oils may be defined by the percentage of solid fat content, determined by NMR serial measurements as described in IUPAC method no. 2.150, 7th edition.

The triglyceride oils are preferably confectionery fats, such as palm oil, cocoa butter or other. Further examples of suitable triglyceride oils are illipe butter, shea butter, kokum butter, sal butter or other natural oils or fractions thereof with a similar solid fat content or melting range. Other examples of such oils are hydrogenated or partly hydrogenated soybean oil, rapeseed oil, cotton oil and sunflower oil or fractions thereof. The triglyceride oils may also be synthetic or semi-synthetic.

The term “confectionary fat” refers to special fats for confectionary applications and is known in the art. Cacao butter is the best known representative of this group, confectionary fats are also often referred to as cacao butter alternatives or cacao butter equivalents, sometimes also as cacao butter replacers or cacao butter substitutes.

The term synthetic or semi-synthetic refers to substances that are not entirely natural and/or obtained by chemical synthesis.

The invention especially refers to the use of compositions wherein the triglyceride oils comprise a fraction of palm oil. This fraction of palm oil may be obtained from commercial palm oil, which may be fractionated to specific mixtures of suitable triglycerides, based on the combination of mainly palmitic, oleic, linoleic and stearic esters of glycerol, respectively.

Preferred fatty acids for use in the invention are therefore selected from the group consisting of palmitic acid, oleic acid, linoleic acid and stearic acid. Even more preferred compositions comprise at least two fatty acids selected from the group consisting of palmitic acid, oleic acid, linoleic acid and stearic acid. Particularly good results were achieved when 20-80%, such as 30-70% of fatty acids were used selected from the group consisting of palmitic and stearic acid, and 80-20%, such as 70-30% fatty acids selected from the group consisting of oleic and linoleic acid. It should be noted that these amounts do not necessarily have to add up to 100%, i.e. they do not necessarily exclude the presence of additional fatty acids such as Lauric acid.

The triglyceride oils may contain at least 90% by weight of triglycerides, such as more than 95% by weight. Also, the content of triglycerides in the palm oil fraction may be 99% or more by weight. The purity can be checked by conventional chromatographic methods, such as thin-layer chromatography or high-performance liquid chromatography. It is preferred that the triglyceride oils utilised in the emulsion are pure and free from unwanted contaminants when used for pharmacological purposes

Any emulsifier may be used in the invention, however, food emulsifiers are preferred. Food emulsifiers are emulsifiers commonly used in food applications and are generally esters composed of a hydrophilic and a lipophilic part. In general, the lipophilic part comprises stearic, palmitic, oleic, or linoleic acid or a combination of said fatty acids. The hydrophilic part generally comprises hydroxyl, carboxyl, or oxyethylene groups.

Examples of families of food-grade emulsifiers are lecithins, mono- and diglycerides, propylene glycol monoesters, lactylated esters, polyglycerol esters, sorbitan esters, ethoxylated esters, succinylated esters, fruit acid esters, acetylated mono and diglycerides, phosphated mono- and diglycerides and sucrose esters. The emulsion of the triglyceride oils can also be obtained when the oils are mixed with suitable foods or food products, making use of the inherent emulsification properties of said foods or food products. Food emulsifiers according to the invention may be able to emulsify more than 20% by weight of the triglyceride oils, preferably more than 40% by weight, giving an emulsion which is still liquid in order to facilitate the processing of a food product in which the emulsion may be incorporated.

A preferred emulsifier of the invention is lecithin, for instance produced from egg yolk, milk, soybean oil, sunflower oil, and rapeseed oil, which consists of a mixture of mainly phospholipids, such as phosphatidylcholine and phosphatidylethanolamine. Lecithin refers in this context to crude mixtures of said phospholipids which are obtained on degumming of the starting materials, and which are commercially available as food emulsifiers.

A particularly preferred emulsifier is a galactolipid-based emulsifier. Galactolipids belong to the group of glycolipids, well known constituents of plant cell membranes. The most important classes of these contain one to four sugars linked glycosidically to diacylglycerol. The two most abundant classes contain one and two galactose units, respectively, and the commonly used nomenclature and abbreviations of these are mono- and digalactosyldiglyceride (MGDG and DGDG), sometimes referred to as galactolipids. Galactolipids, primarily DGDG and DGDG-rich materials, have been investigated and found to be a surface active material of interest in industrial applications such as food, cosmetics, and pharmaceutical products. Galactolipid emulsifiers are described in WO 95/20943 and WO 97/11141. Preferred sources for the galactolipid emulsifiers are cereals and grains, particularly oats.

A preferred aspect of the invention is the use of a composition wherein the triglyceride oils of the invention are combined with palm oil, palmkernel oil or coconut oil.

Particularly good results were obtained when a fractionated oat oil was used as a galactolipid based emulsifier. The invention therefore also relates to the use of a composition wherein the galactolipid based emulsifier was a fractionated oat oil.

Oil-in-water emulsions may be prepared by using the emulsifier either alone or in combination with other amphiphilic compounds, such as co-surfactants. The oil-in-water emulsion may also comprise optional additives known in the art for improving different aspects of the composition, such as flavouring agents, sweeteners, colorants, thickening agents, preservatives, antioxidants, etc.

Oil-in-water emulsions may be prepared by conventional methods. For example, a 30 wt % emulsion of a triglyceride oil in water is prepared by adding the emulsifier to the liquid triglyceride. The continuous phase may be pure water or an aqueous solution containing water-soluble additives such as isotonic agents, sweeteners, flavours, and preservatives. If necessary, the pH of the aqueous phase is then adjusted. The oil phase as well as the aqueous phase are preheated and then the oil phase is added to the aqueous phase under high-shear mixing. The pre-emulsion may then be subjected to high-pressure homogenisation.

The compositions described herein may be administered in enteric or oral doses in order to obtain the body fat mass loss effect and/or the effect of maintenance of blood pressure (for example after a reduction in blood pressure following weight loss). Accordingly, the invention concerns a mixture of a triglyceride oil having a solid fat content at ambient to body temperature and an emulsifier for increasing body fat mass loss in an individual or for maintaining blood pressure in an individual.

The invention also concerns a method for increasing body fat mass loss in an individual or for maintaining blood pressure in an individual, which method comprises the step of administering to the individual an effective amount of a mixture of a triglyceride oil, optionally having a solid fat content at ambient to body temperature, and an emulsifier.

Preferably, the compositions are administered in the form of a food substance. Therefore, the mixture comprising the triglyceride oils plus the emulsifier may be added to solid or semi-solid foods, which then become naturally emulsified to an oil-in-water emulsion on exposure to the fluids of the gastrointestinal tract. The mixture may also contain oil-soluble additives such as antioxidants and flavours. The mixture may also be made into a ready-prepared emulsion which can be added to liquid or semi-liquid foods and drinks.

The invention particularly refers to a food composition wherein the mixture of triglyceride oils and emulsifier of the emulsion comprises 80-99% by weight of triglycerides and 1-20% by weight of emulsifier.

It should be emphasized that the emulsifying capacity of the emulsifier depends on the nature or properties of the emulsifier. The fractionated oat oil mentioned above can without further purification be used as an emulsifier in an amount of 1-20% by weight of the total composition for preparing oil-in-water emulsions of 5-60% by weight of triglycerides. The galactolipid emulsifier of WO 95/20943 should be used in 0.1-5.0% by weight of the total composition for preparing oil-in-water emulsions of 5-80% by weight of triglycerides.

The mixture can be used in formulation of dairy products, such as yogurt, ice cream, margarines, spreads, salad oils and dressings, processed meat products, confectionery, fillings, sauces, soups, fruit drinks, desserts, baby foods, but also nutritional and pharmaceutical supplements. Especially the oily mixture can be used in solid or semi-solid foods such as chocolates, other candies, baked goods and any other appropriate foods.

The invention also refers to the use of a dairy product comprising 1-30% by weight, preferably 2-15% by weight of the oil-in water emulsion. A preferred dairy product, such as a yoghurt, may comprise 4-10% by weight of an emulsion of a triglyceride fraction of palm oil and fractionated oat oil.

In order to obtain the desired effect of body fat mass loss or maintenance of blood pressure, an emulsion of, for example, about 40 wt % may be taken. The emulsion may be in an amount of from about 1 to about 200 ml per serving or meal, alternatively from about 5 to about 100 ml or from about 10 to about 30 ml. The oil component alone, that is the oily mixture, may be used in proportionally smaller quantities.

The mixture described herein may be taken daily for a period of for at least 1 week, at least 2 weeks, at least 4 weeks, at least 8 weeks, at least 12 weeks, at least 16 weeks or longer. The aim of the invention may be achieved when the mixture is taken for a single time period or for multiple time periods interspersed with periods of a diet in which the mixture is not taken, for example a diet of neutral energy balance.

The invention also refers to the use of an oil-in-water emulsion of a triglyceride oil, optionally having a solid fat content at ambient to body temperature, and an emulsifier for the preparation of a pharmacologically active composition for increasing body fat mass loss or for maintaining blood pressure. Accordingly, the invention relates to a method for maintaining blood pressure in an individual, which method comprises the step of administering to an individual a therapeutically effective amount of a mixture of a triglyceride oil, optionally having a solid fat content at ambient to body temperature, and an emulsifier. This is particularly useful after a period of intentional weight loss.

When used in a pharmaceutical composition such a composition may in addition to the oil in-water emulsion comprise a therapeutically active component other than the components according to the invention. Therapeutically active components that may be added include vitamins, minerals and ethical drugs.

The following fats or oils may be used in the mixtures used in the invention. Fractionated palm oil (CPL-Palm oil, LTP Lipid Technologies Provider AB, Karlshamn, Sweden) obtained by fractionation og Akofrite (trade name for a palm oil from Karlshamns AB, Karlshamn, Sweden.

As emulsifiers, the following may be used: Fractionated oat oil (LTP Lipid Technologies Provider AB, Karlshamn, Sweden) comprising about 20% DGDG, and prepared from oats in-accordance with WO 97/11141; or Galactolipids (CPL-Galactolipids, LTP Lipid Technologies Provider AB, Karlshamn, Sweden) comprising about 60% DGDG, and prepared from oats in accordance with WO 95/20943.

The Fractionated palm oil used may have the following fatty acid composition as determined by means of gas-liquid chromatography after alkaline methanolysis: 40-45 wt % palmitic acid, 38-42 wt % oleic acid, 8-10 wt % linoleic acid, and 4-5 wt % stearic acid, the remainder being selected from the group consisting of lauric acid, myristic acid, arachidic acid and palmitoleic acid.

The Fractionated palm oil may have a triglyceride (TG) content of 99.8-100.0 wtB, a solid fat content at 20 and 35 C (N and N35) of 31 and 6, respectively.

The invention is illustrated by the following Examples:

### EXAMPLES

### Example 1

### Fabuless™ Emulsion

Preparation of 40 wt % emulsions with Fractionated palm oil (batch size 300 g).

| Ingredients | wt % |
|---|---|
| Water | 57.5 |
| Fractionated palm oil | 40.0 |
| Fractionated oat oil | 2.5 |

The palm oil is melted at 50° C. and mixed with the fractionated oat oil. The oil phase and the water are preheated to 65-70° C. and then the oil phase is added to the water under high-shear mixing at 15,000 rpm for 4 min. The pre-emulsion is then divided into two parts; one part is homogenized at 400 bar, the other part at 800 bar, both for 6 cycles at 60° C. (Rannie homogenizer, Model Mini-Lab 8.30H, APV Rannie, Denmark).

Both parts of the preparation result in emulsions with a similar cream-like consistency. The average particle size (Z average) is in both cases around 480 nm (Zetasizer 4, Malvern Instruments, UK).

An emulsion prepared as above (herein after called Fabuless™ and marketed by DSM Food Specialties, Delft, Netherlands) can be stored at 2-8° C. until being used as an ingredient in the production of a product.

### Example 2

### Study on Weight Maintenance after a Period of Weight Loss

### Introduction

A study was carried out to investigate the effects of consumption of a meal replacement product with or without Fabuless™ in relation to body weight maintenance and body composition.

The primary objective was to determine the effects of a meal replacement with Fabuless™ on body-weight maintenance after 12 weeks consumption with the same meal replacement without Fabuless™ but with the same fat content.

The secondary objectives were to determine the effects of a meal replacement with or without Fabuless™ on:
fat mass, fat free mass and skeletal mass;
waist and hip circumferences;
body temperature and sagittal measurements;
blood pressure and pulse rate; and
safety.

### Study Design

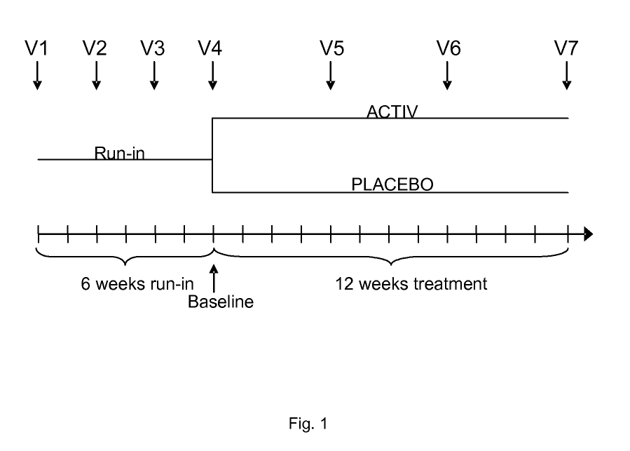

The study was a randomized, double-blind, placebo-controlled, parallel design with 4 to 6 weeks run-in weight loss period and 12 weeks intervention study with voluntary free-living subjects. The double-blindness required that the study foods were very similar as well as packed and labelled equivalently. The study design is shown schematically in FIG. 1 and the schedule of events at each visit is set out in Table 1.

Weight loss was achieved by a 4-6 weeks regimen of Nutrilett Intensive, a commercially available product. Nutrilett Intensive is a VLCD product powder that is mixed with 200 ml of water in a shaker for a complete meal. The subject chose between two flavours: Forest Fruit (112 kcal/bag) and Chocolate (111 kcal/bag). The product was stored, prepared and consumed according to the manufacturer's instructions.

The first two run-in weeks, the subjects were instructed to use 5 bags of Nutrilett Intenstive each day, i.e. 2.3 kJ/day (557/day) in total. The subjects were also obliged to drink a minimum of 2.5 litres of non-calorific beverage each day, such as water/light lemonade/soda, or tea/coffee without milk or sugar. No other foods or drinks were allowed.

From week 3, the subjects used a regime similar to the first two run-in weeks, except that the subjects could eat a regular breakfast and then replace the remaining meals of the day with 4 bags of Nutrilett Intensive. The subjects received information as to how to choose a healthy breakfast, low in fat and sugar and rich in dietary fibre.

For the 12 week intervention study, the subjects returned to their habitual eating patterns and replaced lunch with a Nutrilett Intensive meal (one bag) containing either control or Fabuless™ (LCD meal).

Measurements that took place on the various test days are described below.

### Test Products

The investigational product was a meal replacement (Nutrilett Intensive) with soy bean protein which was consumed by both the treatment groups. The subjects were randomized to an addition of either Fabuless™ or fat from milk (cream, placebo). The fat amount in the placebo was the same as in Fabuless™. The added Fabuless™ or milk fat were served as a drink shot to take together with a portion of Nutrilett Intensive.

The study product, Fabuless™ (42%), and the reference product, placebo, were filled in 15 ml brown glass medicine bottles with screw caps under controlled environmental conditions. The placebo product was cream with an extended shelf-life (Långhållbarhetsgrådde 36%) from Norrmejerier. The bottles were stored in the refrigerator during the study but placed at room temperature 1-2 hours before use. The bag with meal replacement powder, Chocolate or Forest Fruit, was mixed with 200 ml water in a plastic container according to the instructions of the manufacturer and thereafter Fabuless™ or control product was poured in the container with Nutrilett Intensive formulation and shaken for 5 seconds.

Nutritional fact of the study product, Fabuless™, per day:

| | |
|---|---|
| Test product | Fabuless ™ (42%) |
| Total amount | 12.5 g |
| Fat | 5.2 g |
| Protein | 0 |
| Carbohydrate | 0 |
| Total energy | 194 kJ/46 kcal |

Nutritional fact of the placebo product per day:

| | |
|---|---|
| Test product | Cream |
| Total amount | 13.8 g |
| Fat | 5.2 g |
| Protein | 0.4 g |
| Carbohydrate | 0.3 g |
| Total energy | 206 kJ/49 kcal |

### Subjects

Fifty-five females were recruited from the pool of volunteers of the Centrum för Klinisk Prövning av Livsmedel (KPL), Uppsala, Sweden, or via the local newspaper, aged between 18 and 60 with a BMI between 26 and 31 kg/m². They were in good health, with a blood pressure not exceeding 160/105 mmHg, not using medication. Only subjects with a weight loss more than 5% after 6 weeks or 9% after 4 weeks were allowed to continue into the intervention study.

The subjects who fulfilled the inclusion criteria after the run-in period were randomized into the intervention study with active or placebo treatment. Test products were distributed at each visit together with a study diary to fill in and sign every day they consumed the investigational products.

### Measurements

Weight, waist, hip and sagittal measurements of the abdomen (SAG), height, body temperature, blood pressure, pulse, physical activity, bioelectrical impedance and caliper were measured at every visit (see FIG. 1 and Table 1) in a fasted state. Blood samples for clinical chemistry, glucose haematology, urine were taken before the run-in, at the baseline and at the end of the study.

### Body Weight and Height

Body weight was assessed at all visits. Body weight was measured in kilograms (kg) and the subjects were scaled with indoor clothes without shoes, wallet or keys. The scale used was a CL-300 BMI, Carl Liden, Gothenburg, Sweden. The same scale was used throughout the study. Height was measured in centimetres (cm) without shoes.

### Waist and Hip Measurement

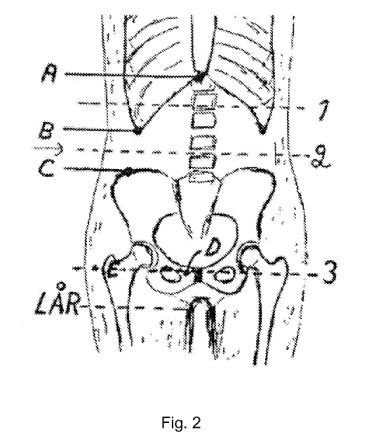

Waist and hip measurements were performed at all visits. The subject was standing. The study nurse wrapped a measuring-tape around the subject's waist. The measuring-tape was placed between the iliac crest and the lower part of the breastbone (see number 2 or arrow in FIG. 2). The subject's clothes, except for underwear, were removed from waist and hip during the measurements. The study nurse asked the subject to exhale before the waist was measured. Hip was measured at number 3 in FIG. 2.

### Sagittal Measurement (SAG)

Sagittal measurement of abdomen was performed at all visits. SAG was measured with the subject lying on their back on a flat, hard surface and the distance between the surface and the highest point of the abdomen, at the subject's iliac crest. The subject's clothes, except for underwear, were removed from the abdomen during the measurements.

### Blood Pressure and Pulse Rate

Blood pressure and pulse were assessed at all visits. Blood pressure and pulse rate were measured oscillometrically (Omron M4-I, Omron Healthcare Europe B.V., Hoofdorp, The Netherlands) in a sitting position in the right arm after a resting period of 3 to 5 min. Totally 3 measurements with 2 to 5 min in between were performed and an average of these was calculated.

### Body Temperature

Body temperature was measured at visit 2 to 7. The same thermometer was used during the study. Body temperature was measured rectally by Terumo Electronic Fever Thermometer C402.

### Caliper Measurements

Caliper measurements were performed at all visits. For a reproducible result, all study personnel (the same nurse) performing caliper measurements were trained in the technique. Measurements were performed at four different locations on the body: biceps, triceps, subscapular and suprailiac. Measurements were made according to specific “Caliper measurement instructions”.

### Bioelectrical Impedance

Bioelectrical impedance was performed at all visits. The subject should be fasting to get a reliable result from the measurement. The measurement was performed with the subject lying on a horizontal flat wooden surface with a Xitron Technologies (Hydra ECF/ICF) according to the apparatus instructions.

The caliper and bioelectric impedance measurements were the basis for calculation of body mass, body fat free mass and skeletal muscle mass according to Forslund et al. Evaluation of modified multicompartment models to calculate body composition in healthy males. Am. Clin. Nutr. 1996, 6, 856-862.

### Statistical Methods

For the statistical analysis, SPSS version 15.0 for windows was used in this study. The statistical analysis was conducted by KPL.

Student's t-test, this test is two-sided and performed at 5% significant level. The treatment differences were analysed with a paired t-test.

Descriptive statistics were calculated for the parameters of body fat mass and body muscle mass, by using box-and-whiskers-plot. Boxplots allow comparing each group using a five-number summary: the median, the 25th and 75th percentiles, and the minimum and maximum observed values that are not statistically outliers. Outliers and extreme values are given special attention. The heavy black line inside each box marks the 50th percentile, or median, of that distribution. The lower and upper hinges, or box boundaries, mark the 25th and 75th percentiles of each distribution, respectively. Whiskers appear above and below the hinges.

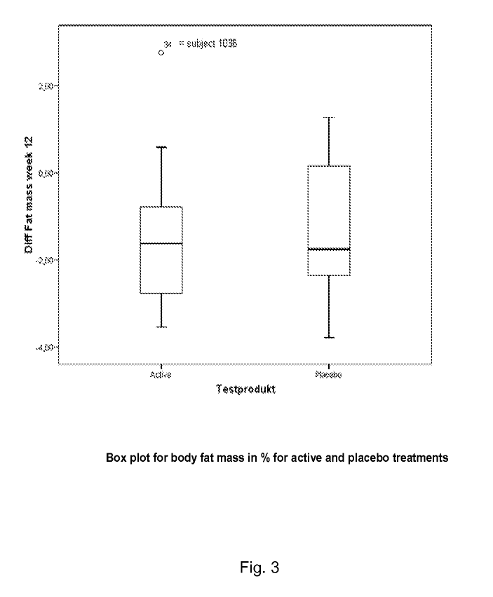

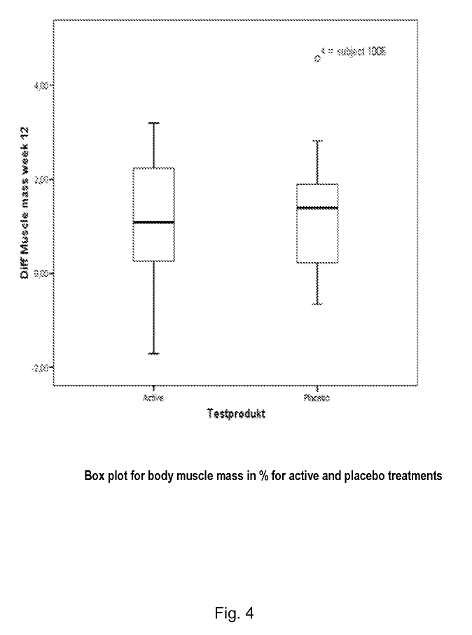

Whiskers are vertical lines ending in horizontal lines at the largest and smallest observed values that are not statistical outliers. Outliers are identified with an 0 (see FIGS. 3 and 4).

### Results

### Study Subjects

The recruitment procedure resulted in 79 potentially eligible female subjects to come for a screening visit. Out of these subjects, 54 were found eligible to the run-in period and thereafter 46 subjects were randomized into active or placebo treatment. Subject distribution showing 23 subjects on each treatment and 43 subjects were found eligible to do statistical analyses on. One subject was withdrawn due to her own decision and two subjects had compliance lower than 70% and were therefore not included into the statistical analyses.

The screening period was during week 31, 2006 and the intervention period was then running during weeks 32-49, 2006 with 6 weeks run-in period and 12 weeks intervention of the study product.

In total 46 subjects were randomized to one of the two treatment sequences active or placebo. The distribution of subjects in each treatment group was 23 consumed active and 23 females consumed placebo. The number of subjects completing the study was 45 out of 46 randomized subjects.

After randomization one subject withdrew from the study due to her own decision.

The relative compliance was 90.8% for the active treatment and 91.4% for the placebo treatment. Two subjects had compliance less then 70% and were therefore not included into statistical analyses. In total 43 subjects were included into the statistical analyses.

### Background Characteristics

Definition of efficacy population was subjects with no major protocol deviation and a compliance of at least 70%.

The mean age was 46.4 years (SD 9.7) for active and 49.4 years (SD 7.8) for placebo. The mean BMI at run-in was 28.2 (SD 1.4) for active and 28.3 (SD 1.6) for placebo treatment group.

No big differences were observed between the treatment groups in respect of blood pressure (systolic and diastolic pressure). Pulse rate, waist, hip sagittal, body fat mass, fat free mass and skeletal muscle mass were registered for the two treatment groups and, again, no big differences were shown between treatment groups.

Baseline (visit 4) characteristics showed a somewhat higher blood pressure for the group that was on active treatment both in systolic (120.7) compared to placebo (115.9) and in the diastolic blood pressure for active treatment (79.7) compared to placebo (74.7). The BMI was similar between the treatment groups, but showed a clear reduction compared to the run-in characteristics. The same pattern was shown for the other baseline characteristics (pulse rate, waist, hip sagittal, body fat mass, fat free mass and skeletal muscle mass) with no big differences between treatment groups, but a clear reduction compared to run-in characteristics.

The energy intake according to compliance was about 42 kcal per day for active product and almost 45 kcal for placebo treatment.

Two subjects with randomization numbers 1006 (placebo) and 1036 (active) were considered as outliers after calculations made on body fat mass (1036) and muscle mass (1006). This is clearly demonstrated in the boxplots set out in FIGS. 2 and 3.

The results presented herein are therefore based on efficacy variables without these two outliers.

### Body Weight

The change from baseline in body weight after 12 weeks consumption of Fabuless™ and placebo with meal replacement showed statistically significant differences (see Table 2). When comparing the body weight of the females in the two treatments no statistical differences were shown (see Table 3).

Change in body weight from baseline after 4 and 8 weeks after consumption of Fabuless™ with meal replacement showed statistical differences at both visits (see Table 3). No differences were seen for the placebo treatment after 4 or 8 weeks. When comparing the change in body weights between treatments no differences could be seen (see Table 3).

### Fat Mass, Fat Free Mass and Skeletal Mass

Change in body fat mass, fat free mass and muscle mass from baseline (visit 4) and after 4, 8 and 12 weeks consumption of Fabuless™ or placebo with meal replacement showed statistically significant differences at all visits for both treatments (see Tables 4 and 5).

The comparison between treatment also showed statistically significant differences at week 8 (p-value 0.023) for body fat mass and body fat free mass (Tables 6 and 7). There is a significant difference on body fat mass and fat free mass between treatments with a 1-tailed t-test at week 8 and 12.

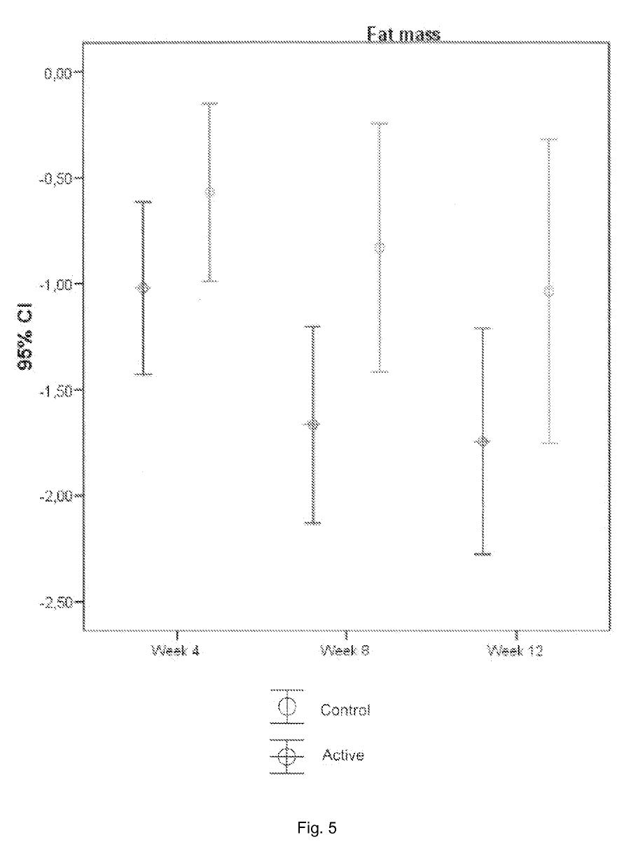

This was further confirmed for the 12 week treatment period when the area under the curve was calculated and compared between the two treatments for body fat mass (see Tables 8 to 10). This is also demonstrated clearly in FIG. 5 from the shape of the slope, which is steeper for the active treatment.

### Hip and Waist

Changes in waist and hip circumferences from baseline (visit 4) and after 4, 8 and 12 weeks consumption of meal replacement with Fabuless™ showed statistically significant differences after 12 weeks in waist circumference and statistically significant difference at all visits in hip circumference (see Table 11). Change in waist circumference after consumption of meal replacement with placebo showed no differences at any visit but a statistically significant difference in hip circumference after 8 and 12 weeks. The comparison between treatments did not show any differences (see Tables 6 and 7).

### Systolic Blood Pressure

No change from baseline in systolic blood pressure was noticed in the active group but a significant increase was observed at all visits in the placebo group (Table 12). A significant difference was seen between the treatments at week 8.

### Conclusions

After a weight loss phase, the participants in this study continued to replace one meal (lunch) with a meal replacement product containing Fabuless™ or cream (LCD). Thus, on a negative energy balance during the intervention phase, the subjects continued to lose body fat mass and to a significantly higher degree in the Fabuless™ group (a statistically significant difference, p=0.023, could be observed after 8 and 12 weeks consumption).

The systolic blood pressure was decreased as expected during the weight loss period. Despite the same amount of fat in the two test products used during the intervention, the systolic blood pressure was maintained in the Fabuless™ group, but significantly increased from baseline in the placebo group.

### Example 3

### Additional Statistical Analysis of Caliper Measurements

Further statistical analysis was carried out on the data of skinfold thickness measured by caliper, an established method for calculation of body composition over time. This evaluation was made at baseline, and after 4, 8 and 12 weeks of treatment and calculation of body fat mass was made according to the method of Durnin and Wormersly (Brit J. Nutr. (1974) 32, 77).

An analysis of variance (ANOVA) was used for the statistical evaluation. The decrease in body fat mass in the active group was significantly more with respect to placebo p=0.0108 (see Table 13).

### Tables

TABLE 1
Schedule of events

| | Type of action: | | | | | | | |
|---|---|---|---|---|---|---|---|---|
| | | Run-in | | | Baseline | Evaluation | Evaluation | Evaluation |
| | | Visit number: | | | | | | |
| | | Visit 1 | Visit 2 | Visit 3 | Visit 4 | Visit 5 | Visit 6 | Visit 7 |
| | | Week | | | | | | |
| | | Week | Week | Week | | | | Week |
| | | −6 | −4 | −2 | Week 1 | Week 5 | Week 9 | 13 |
| | | Days (±3 days) | | | | | | |
| | Telephone | Day | Day | Day | | Day | Day | Day |
| | Screening | −43 | −29 | −15 | Day 1 | 29 | 57 | 85 |
| Incl./Excl. criteria | x¹ | | | | | | | |
| Weight, Waist, Hip | | x | x | x | x | x | x | x |
| and SAG | | | | | | | | |
| Height | | x | | | | | | |
| BMI | x | x | x | x | x | x | x | x |
| Body temperature | | | x | x | x | x | x | x |
| Blood pressure, | | x | x | x | x | x | x | x |
| pulse rate | | | | | | | | |
| Physical activity | | x | x | x | x | x | x | x |
| Bioelectrical | | x | x | x | x | x | x | x |
| impedance and | | | | | | | | |
| caliper | | | | | | | | |
| Signed consents and | | x | | | | | | |
| subject information | | | | | | | | |
| Clinical chemistry | | x | | | x | | | x |
| (P-ALAT, P-γGT, P- | | | | | | | | |
| Createnine, P-Na, P- | | | | | | | | |
| K, P-Triglycerides, | | | | | | | | |
| P-Apolipoprotein | | | | | | | | |
| A1 and B) | | | | | | | | |
| Diabetes (fP- | | x | | | x | | | x |
| glucose, B-HbA1_{c}) | | | | | | | | |
| Tyreoidea (S-TSH) | | x | | | x | | | |
| Hematology (B-Hb) | | x | | | x | | | x |
| Urine (protein, | | x | | | x | | | x |
| glucose) | | | | | | | | |
| Randomisation | | | | | x | | | |
| Study diary | | x | x | x | x | x | x | x |
| AE/SAE | | | x | x | x | x | x | x |
| Concomitant med | | x² | x | x | x | x | x | x |
| Distribution of food | | x | x | x | x | x | x | |
| (Nutrilett) | | | | | | | | |
| Distribution of | | | | | x | x | x | |
| investigational | | | | | | | | |
| products | | | | | | | | |

¹The study nurse will ask the subject if he/she has this information
²Concurrent medication will be taken at visit 1.

TABLE 2
Summary results of sagittal measurement and weight per treatment
sequence. Subject 1006 and 1036 excluded.

| | | | Run-in | Baseline | Week 4 | Week 8 | Week 12 |
|---|---|---|---|---|---|---|---|
| Sagittal (SAG) | Active | n | 21 | 21 | 21 | 21 | 21 |
| (cm) | | mean | 22.0 | 20.0 | 19.8 | 19.7 | 20.0 |
| | | SD | 1.3 | 1.1 | 1.2 | 1.1 | 1.1 |
| | | median | 22.0 | 20.0 | 19.5 | 20.0 | 19.5 |
| | | min | 19.0 | 17.0 | 17.0 | 17.0 | 18.0 |
| | | max | 24.2 | 21.5 | 22.0 | 22.0 | 23.0 |
| | Placebo | n | 20 | 20 | 20 | 19* | 20 |
| | | mean | 22.0 | 19.6 | 19.5 | 19.4 | 19.4 |
| | | SD | 1.9 | 1.3 | 1.2 | 1.4 | 1.3 |
| | | median | 21.8 | 19.5 | 19.0 | 19.0 | 19.3 |
| | | min | 18.5 | 17.5 | 18.0 | 17.0 | 17.0 |
| | | max | 26.2 | 21.5 | 22.0 | 22.0 | 22.0 |
| Weight | Active | n | 21 | 21 | 21 | 21 | 21 |
| (kg) | | mean | 79.5 | 72.9 | 72.2 | 71.9 | 71.7 |
| | | SD | 6.2 | 5.5 | 5.9 | 5.8 | 5.6 |
| | | median | 77.6 | 71.8 | 69.9 | 69.8 | 70.7 |
| | | min | 68.5 | 63.4 | 63.0 | 61.9 | 61.6 |
| | | max | 91.1 | 84.2 | 85.8 | 85.2 | 83.8 |
| | Placebo | n | 20 | 20 | 20 | 19* | 20 |
| | | mean | 78.6 | 71.1 | 70.6 | 70.4 | 69.9 |
| | | SD | 8.3 | 7.0 | 6.9 | 7.2 | 7.0 |
| | | median | 78.7 | 71.2 | 69.7 | 70.3 | 70.0 |
| | | min | 63.2 | 59.4 | 58.5 | 58.7 | 57.9 |
| | | max | 95.0 | 84.3 | 82.1 | 83.3 | 82.7 |

Subject No's 1004 are not included (withdrawal)
Subject No's 1001 and 1003 are excluded due to less than 70% compliance.
*Subject No's 1007 missing data from this visit.

TABLE 3
Change from baseline in sagittal measurement and weight per treatment
sequence. Subject 1006 and 1036 excluded.

| | | | Week 4 | Week 8 | Week 12 |
|---|---|---|---|---|---|
| Dif. Sagittal (SAG) | Active | n | 21 | 21 | 21 |
| (cm) | | mean | −0.19 | −0.26 | 0.00 |
| | | SD | 0.54 | 0.58 | 0.89 |
| | | median | 0.00 | 0.00 | 0.00 |
| | | min | −1.50 | −1.50 | −1.00 |
| | | max | 0.50 | 1.00 | 2.50 |
| | | Prt | 0.119 | 0.053 | 1.000 |
| | Placebo | n | 20 | 19* | 20 |
| | | mean | −0.13 | −0.11 | −0.20 |
| | | SD | 0.58 | 0.57 | 0.57 |
| | | median | 0.00 | 0.00 | −0.25 |
| | | min | −1.00 | −1.00 | −1.50 |
| | | max | 1.00 | 1.00 | 1.00 |
| | | Prt | 0.349 | 0.429 | 0.134 |
| Diff. Weight | Active | n | 21 | 21 | 21 |
| (kg) | | mean | −0.77 | −1.06 | −1.19 |
| | | SD | 1.53 | 1.76 | 1.96 |
| | | median | −0.70 | −0.80 | −0.90 |
| | | min | −3.30 | −4.60 | −4.20 |
| | | max | 1.80 | 1.50 | 3.00 |
| | | Prt | 0.033 | 0.012 | 0.012 |
| | Placebo | n | 20 | 19* | 20 |
| | | mean | −0.51 | −0.64 | −1.16 |
| | | SD | 1.32 | 1.52 | 1.97 |
| | | median | −0.85 | −0.90 | −1.40 |
| | | min | −2.20 | −2.90 | −5.00 |
| | | max | 2.20 | 2.70 | 3.80 |
| | | Prt | 0.104 | 0.084 | 0.016 |

Subject No's 1004 are not included (withdrawal)
Subject No's 1001 and 1003 are excluded due to less than 70% compliance.
*Subject No's 1007 missing data from this visit.

TABLE 4
Change from baseline in body fat mass and body fat free mass per
treatment sequence. Subject 1006 and 1036 excluded.

| | | | Week 4 | Week 8 | Week 12 |
|---|---|---|---|---|---|
| Diff. Body fat mass | Active | n | 21 | 21 | 21 |
| (% of body weight) | | mean | −1.02 | −1.67 | −1.74 |
| | | SD | 0.90 | 1.02 | 1.17 |
| | | median | −0.78 | −1.59 | −1.64 |
| | | min | −3.14 | −3.40 | −3.54 |
| | | max | 1.07 | −0.29 | 0.59 |
| | | Prt | <0.0001 | <0.0001 | <0.0001 |
| | Placebo | n | 20 | 18* | 20 |
| | | mean | −0.57 | −0.83 | −1.04 |
| | | SD | 0.89 | 1.18 | 1.53 |
| | | median | −0.81 | −1.01 | −1.48 |
| | | min | −1.71 | −2.53 | −2.78 |
| | | max | 1.55 | 2.03 | 2.30 |
| | | Prt | 0.0102 | 0.0081 | 0.0070 |
| Body fat free mass | Active | n | 21 | 21 | 21 |
| (% of body weight) | | mean | 1.02 | 1.67 | 1.74 |
| | | SD | 0.90 | 1.02 | 1.17 |
| | | median | 0.78 | 1.59 | 1.64 |
| | | min | −1.07 | 0.29 | −0.59 |
| | | max | 3.14 | 3.40 | 3.54 |
| | | Prt | <0.0001 | <0.0001 | <0.0001 |
| | Placebo | n | 20 | 18* | 20 |
| | | mean | 0.57 | 0.83 | 1.04 |
| | | SD | 0.89 | 1.18 | 1.53 |
| | | median | 0.81 | 1.01 | 1.48 |
| | | min | −1.55 | −2.03 | −2.30 |
| | | max | 1.71 | 2.53 | 2.78 |
| | | Prt | 0.0102 | 0.0081 | 0.0070 |

Subject No's 1004 are not included (withdrawal)
Subject No's 1001 and 1003 are excluded due to less than 70% compliance.
*Subject No's 1007, 1024 are missing data from this visit.

TABLE 5
Change from baseline in body muscle mass per treatment sequence.
Subject 1006 and 1036 excluded.

| | | | Week 4 | Week 8 | Week 12 |
|---|---|---|---|---|---|
| Diff. Body | Active | n | 21 | 21 | 21 |
| muscle mass | | mean | 0.72 | 1.28 | 1.31 |
| (% of body weight) | | SD | 0.78 | 1.08 | 1.27 |
| | | median | 0.38 | 1.32 | 1.16 |
| | | min | −0.65 | −0.64 | −1.71 |
| | | max | 2.41 | 3.58 | 3.20 |
| | | Prt | 0.0004 | <0.0001 | 0.0001 |
| | Placebo | n | 20 | 18* | 20 |
| | | mean | 0.81 | 0.94 | 1.18 |
| | | SD | 0.85 | 1.24 | 1.00 |
| | | median | 0.86 | 0.84 | 1.42 |
| | | min | −0.67 | −1.35 | −0.66 |
| | | max | 2.60 | 3.23 | 2.82 |
| | | Prt | 0.0004 | 0.0052 | <0.0001 |

Subject No's 1004 are not included (withdrawal)
Subject No's 1001 and 1003 are excluded due to less than 70% compliance.
*Subject No's 1007, 1024 are missing data from this visit.

TABLE 6
Difference in pvalue, 2-tailed. Subject 1006 and 1036 excluded.

| | Week 4 | Week 8* | Week 12 |
|---|---|---|---|
| Diff. Systolic blood pressure (mm Hg) | 0.786 | 0.066 | 0.227 |
| Diff. Diastolic blood pressure (mm Hg) | 0.841 | 0.345 | 0.549 |
| Diff. Pulse rate (beats/min) | 0.837 | 0.784 | 0.606 |
| Diff. Body temperature (° C.) | 0.587 | 0.420 | 0.134 |
| Diff. Waist (cm) | 0.945 | 0.332 | 0.261 |
| Diff. Hip (cm) | 0.475 | 0.924 | 0.864 |
| Diff. Body fat mass (% of body weight) | 0.113 | 0.023 | 0.102 |
| Body fat free mass (% of body weight) | 0.113 | 0.023 | 0.102 |
| Diff. Body muscle mass (% of body | 0.726 | 0.364 | 0.718 |
| weight) | | | |
| Dif. Sagittal (SAG) (cm) | 0.710 | 0.396 | 0.401 |
| Diff. Weight (kg) | 0.563 | 0.426 | 0.967 |

Subject No's 1004 are not included (withdrawal)
Subject No's 1001 and 1003 are excluded due to less than 70% compliance.
*Subject No's 1007 missing data from this visit.

TABLE 7
Difference in pvalue, 1-tailed. Subject 1006 and 1036 excluded.

| | Week 4 | Week 8* | Week 12 |
|---|---|---|---|
| Diff. Systolic blood pressure (mm Hg) | 0.393 | 0.033 | 0.114 |
| Diff. Diastolic blood pressure (mm Hg) | 0.421 | 0.173 | 0.275 |
| Diff. Pulse rate (beats/min) | 0.419 | 0.392 | 0.303 |
| Diff. Body temperature (° C.) | 0.294 | 0.210 | 0.067 |
| Diff. Waist (cm) | 0.473 | 0.166 | 0.131 |
| Diff. Hip (cm) | 0.238 | 0.462 | 0.432 |
| Diff. Body fat mass (% of body weight) | 0.057 | 0.012 | 0.051 |
| Body fat free mass (% of body weight) | 0.057 | 0.012 | 0.051 |
| Diff. Body muscle mass (% of body | 0.363 | 0.182 | 0.359 |
| weight) | | | |
| Dif. Sagittal (SAG) (cm) | 0.355 | 0.198 | 0.201 |
| Diff. Weight (kg) | 0.282 | 0.213 | 0.484 |

Subject No's 1004 are not included (withdrawal)
Subject No's 1001 and 1003 are excluded due to less than 70% compliance.
*Subject No's 1007 missing data from this visit.

TABLE 8
Area under the curve (auc) for body fat mass (% of body weight). Subject
1006 and 1036 excluded.

| Testprodukt | | Week 0-4 | Week 4-8 | Week 8-12 | Week 0-8 | Week 0-12 |
|---|---|---|---|---|---|---|
| Active | N | 21 | 21 | 21 | 21 | 21 |
| | Mean | −0.51 | −1.34 | −1.71 | −1.86 | −3.56 |
| | Std. Deviation | 0.45 | 0.84 | 1.03 | 1.25 | 2.16 |
| | Median | −0.39 | −1.37 | −1.72 | −1.81 | −4.06 |
| | Minimum | −1.57 | −3.27 | −3.47 | −4.84 | −8.32 |
| | Maximum | 0.54 | 0.24 | 0.00 | 0.78 | 0.44 |
| Placebo | N | 20 | 18* | 18* | 18* | 18* |
| | Mean | −0.28 | −0.70 | −1.03 | −0.98 | −2.01 |
| | Std. Deviation | 0.45 | 0.99 | 1.24 | 1.44 | 2.58 |
| | Median | −0.41 | −0.87 | −1.47 | −1.27 | −2.57 |
| | Minimum | −0.86 | −1.91 | −2.44 | −2.75 | −4.92 |
| | Maximum | 0.78 | 1.79 | 1.65 | 2.56 | 4.21 |

Subject No's 1004 are not included (withdrawal)
Subject No's 1001 and 1003 are excluded due to less than 70% compliance.
*Subject No's 1007, 1024 are missing data from this visit.

TABLE 9
Area under the curve (auc) for body muscle mass (% of body weight).
Subject 1006 and 1036 excluded.

| Testprodukt | | Week 0-4 | Week 4-8 | Week 8-12 | Week 0-8 | Week 0-12 |
|---|---|---|---|---|---|---|
| Active | N | 21 | 21 | 21 | 21 | 21 |
| | Mean | 0.36 | 1.00 | 1.30 | 1.36 | 2.65 |
| | Std. Deviation | 0.39 | 0.79 | 1.05 | 1.12 | 2.03 |
| | Median | 0.19 | 0.73 | 1.16 | 0.82 | 1.79 |
| | Minimum | −0.32 | −0.11 | −0.28 | −0.09 | −0.11 |
| | Maximum | 1.20 | 2.41 | 2.78 | 3.61 | 6.23 |
| Placebo | N | 20 | 18* | 18* | 18* | 18* |
| | Mean | 0.40 | 0.89 | 1.02 | 1.31 | 2.33 |
| | Std. Deviation | 0.42 | 0.97 | 1.08 | 1.38 | 2.33 |
| | Median | 0.43 | 0.81 | 1.12 | 1.37 | 2.21 |
| | Minimum | −0.33 | −1.01 | −0.56 | −1.34 | −1.87 |
| | Maximum | 1.30 | 2.38 | 3.02 | 3.43 | 6.18 |

Subject No's 1004 are not included (withdrawal)
Subject No's 1001 and 1003 are excluded due to less than 70% compliance.
* Subject No's 1007, 1024 are missing data from this visit.

TABLE 10
Difference in pvalue (2-tailed) between treatment groups for area under
the curve (auc). Subject 1006 and 1036 excluded.

| | | Week | Week | Week | |
|---|---|---|---|---|---|
| | Week 0-4 | 4-8 | 8-12 | 0-8 | Week 0-12 |
| AUC. Body fat mass | 0.113 | 0.035 | 0.068 | 0.050 | 0.048 |
| (% of body weight) | | | | | |
| AUC. Body muscle mass | 0.726 | 0.694 | 0.427 | 0.896 | 0.641 |
| (% of body weight) | | | | | |

Subject No's 1004 are not included (withdrawal)
Subject No's 1001 and 1003 are excluded due to less than 70% compliance.
*Subject No's 1007, 1024 are missing data from this visit.

TABLE 11
Change from baseline in waist and hip per treatment sequence. Subject
1006 and 1036 excluded.

| | | | Week 4 | Week 8 | Week 12 |
|---|---|---|---|---|---|
| Diff. Waist | Active | n | 21 | 21 | 21 |
| (cm) | | mean | 0.10 | −1.10 | −1.38 |
| | | SD | 2.55 | 2.72 | 2.58 |
| | | median | 1.00 | 0.00 | −1.00 |
| | | min | −6.00 | −8.00 | −8.00 |
| | | max | 4.00 | 2.00 | 3.00 |
| | | Prt | 0.866 | 0.080 | 0.023 |
| | Placebo | n | 20 | 19* | 20 |
| | | mean | 0.15 | −0.26 | −0.45 |
| | | SD | 2.50 | 2.62 | 2.65 |
| | | median | 0.00 | 0.00 | 0.00 |
| | | min | −5.00 | −5.00 | −6.00 |
| | | max | 5.00 | 5.00 | 5.00 |
| | | Prt | 0.791 | 0.667 | 0.456 |
| Diff. Hip | Active | n | 21 | 21 | 21 |
| (cm) | | mean | −0.76 | −1.05 | −1.43 |
| | | SD | 1.51 | 1.86 | 2.29 |
| | | median | −1.00 | −1.00 | −2.00 |
| | | min | −3.00 | −5.00 | −6.00 |
| | | max | 1.00 | 2.00 | 2.00 |
| | | Prt | 0.032 | 0.018 | 0.010 |
| | Placebo | n | 20 | 19* | 20 |
| | | mean | −0.40 | −1.11 | −1.55 |
| | | SD | 1.70 | 1.94 | 2.21 |
| | | median | −0.50 | −1.00 | −2.00 |
| | | min | −2.00 | −4.00 | −5.00 |
| | | max | 4.00 | 3.00 | 3.00 |
| | | Prt | 0.305 | 0.023 | 0.005 |

Subject No's 1004 are not included (withdrawal)
Subject No's 1001 and 1003 are excluded due to less than 70% compliance.
*Subject No's 1007 missing data from this visit.

TABLE 12
Change from baseline in blood pressure per treatment
sequence. Subject 1006 and 1036 excluded.

| | | | Week 4 | Week 8 | Week 12 |
|---|---|---|---|---|---|
| Diff. Systolic blood | Active | n | 21 | 21 | 21 |
| pressure (mmHg) | | mean | 3.3 | −0.1 | 0.9 |
| | | SD | 7.7 | 8.4 | 11.2 |
| | | median | 4.0 | 0.7 | −0.3 |
| | | min | −10.7 | −17.0 | −13.3 |
| | | max | 17.7 | 16.0 | 31.0 |
| | | Prt | 0.63 | 0.949 | 0.727 |
| | Placebo | n | 20 | 19* | 20 |
| | | mean | 4.0 | 5.6 | 4.4 |
| | | SD | 8.5 | 10.7 | 6.7 |
| | | median | 4.8 | 6.0 | 3.3 |
| | | min | −11.3 | −8.7 | −5.3 |
| | | max | 17.0 | 27.7 | 17.3 |
| | | Prt | 0.049 | 0.034 | 0.008 |
| Diff. Diastolic blood | Active | n | 21 | 21 | 21 |
| pressure (mmHg) | | mean | 1.9 | −1.1 | 0.9 |
| | | SD | 3.9 | 4.4 | 6.2 |
| | | median | 1.7 | −1.7 | 1.3 |
| | | min | −6.0 | −12.3 | −9.0 |
| | | max | 10.0 | 4.3 | 14.3 |
| | | Prt | 0.036 | 0.250 | 0.529 |
| | Placebo | n | 20 | 19* | 20 |
| | | mean | 1.6 | 0.6 | 2.1 |
| | | SD | 5.2 | 7.0 | 6.2 |
| | | median | 1.8 | 0.7 | 2.5 |
| | | min | −10.0 | −16.0 | −13.0 |
| | | max | 9.3 | 10.0 | 14.0 |
| | | Prt | 0.182 | 0.710 | 0.157 |

Subject No's 1004 are not included (withdrawal)
Subject No's 1001 and 1003 are excluded due to less than 70% compliance.
*Subject No's 1007 missing data from this visit.

TABLE 13
Summary of body fat mass (% of body weight) measured with caliper for
Example 3

| | | | Baseline | Week 4 | Week 8 | Week 12 |
|---|---|---|---|---|---|---|
| Body fat | Active | n | 22 | 22 | 22 | 22 |
| mass | | Mean | 40.39 | 39.29 | 38.42 | 38.66 |
| (% of body | | SD | 3.17 | 3.01 | 3.13 | 3.17 |
| weight) | Placebo | n | 21 | 21 | 19 | 21 |
| | | Mean | 40.0 | 39.45 | 38.79 | 39.17 |
| | | SD | 3.29 | 3.20 | 3.23 | 3.64 |

1. Use of a mixture comprising a triglyceride oil and an emulsifier for increasing body fat mass loss in an individual.

2. Use of a mixture comprising a triglyceride oil and an emulsifier for the preparation of a pharmacologically active composition for increasing body fat mass loss in an individual.

3. Use of a mixture comprising a triglyceride oil and an emulsifier for maintaining blood pressure in an individual.

4. Use of a mixture comprising a triglyceride oil and an emulsifier for the preparation of a pharmacologically active composition for maintaining blood pressure in an individual.

5. Use according to claim 1, wherein the triglyceride oil has a solid fat content at ambient to body temperature.

6. Use according to claim 1, wherein the increase in body fat mass loss or maintenance of blood pressure takes place after weight loss.

7. Use according to claim 6, wherein the weight loss is at least about 5%.

8. Use according to claim 1, wherein the individual is subject to a negative energy balance.

9. Use to claim 1, wherein the individual is subject to a meal replacement regime.

10. Use according to claim 1, wherein the triglyceride oil is a confectionary fat.

11. Use according to claim 10, wherein the confectionary fat is selected from the group consisting of palm oil, cocoa butter, illipe butter, shea butter, kokum butter, sal butter, hydrogenated or partly hydrogenated soybean oil, rapeseed oil, cotton oil, sunflower oil or fractions thereof or other natural oils or fractions thereof with a similar solid fat content or melting range.

12. Use according to claim 1, wherein the triglycerides are obtained from fractionated palm oil.

13. Use according to claim 12 wherein the triglycerides are palmitic, oleic, linoleic or stearic esters of glycerol.

14. Use according to claim 1, wherein the triglycerides are synthetic or semi-synthetic.

15. Use according to claim 1, wherein the emulsifier is a food emulsifier.

16. Use according to claim 15, wherein the food emulsifier comprises an ester composed of a hydrophilic and a lipophilic part.

17. Use according to claim 16, wherein the lipophilic part is composed of stearic, palmitic, oleic, or linoleic acid or a combination of said fatty acids.

18. Use according to claim 16, wherein the hydrophilic part comprises hydroxyl, carboxyl, or oxyethylene groups.

19. Use according to claim 15, wherein the food emulsifier comprises a lecithin, a mono- and diglyceride, a propylene glycol monoester, a lactylated ester, a polyglycerol ester, a sorbitan ester, an ethoxylated ester, a succinylated ester, a fruit acid ester, an acetylated mono and diglyceride, a phosphated mono- and diglyceride or a sucrose ester.

20. Use according to claim 19, wherein the food emulsifier comprises lecithin produced from egg yolk, milk, soybean oil, sunflower oil, or rapeseed oil.

21. Use according to claim 1, wherein the emulsifier comprises one or more galactolipids.

22. Use according to claim 21, wherein the emulsifier is a galactolipid.

23. Use according to claim 21, wherein the galactolipid contains one to four sugars linked glycosidically to diacylglycerol.

24. Use according to claim 21, wherein the galactolipid is a mono or digalactosyldiglyceride.

25. A mixture comprising a triglyceride oil, optionally having a solid fat content at ambient to body temperature, and an emulsifier for increasing body fat mass loss in an individual.

26. A mixture comprising a triglyceride oil, optionally having a solid fat content at ambient to body temperature, and an emulsifier maintaining blood pressure in an individual.

27. A mixture according to claim 26, wherein the triglyceride oil is a confectionary fat.

28. A method for increasing body fat mass loss in an individual, which method comprises the step of administering to the individual an effective amount of a mixture comprising a triglyceride oil, optionally having a solid fat content at ambient to body temperature, and an emulsifier.

29. A method for maintaining blood pressure in an individual, which method comprises the step of administering to the individual an effective amount of a mixture comprising a triglyceride oil, optionally having a solid fat content at ambient to body temperature, and an emulsifier.

30. A method according to claim 29, wherein the triglyceride oil is a confectionary fat.

31. A product comprising:
a mixture comprising a triglyceride oil, optionally having a solid fat content at ambient to body temperature, and an emulsifier; and
a meal replacement product.

32. A product according to claim 31, wherein the triglyceride oil is a confectionary fat.

33. A product according to claim 31, wherein the meal replacement product is in the form of a bar, a powder or a liquid.

34. A product according to claim 31, wherein the meal replacement product provides from about 100 to about 400 kcalories per serving.

35. A product according to claim 31, wherein the mixture and the meal replacement product are for simultaneous, separate or sequential use.

36. A product according to claim 31, wherein the product is for increasing body fat mass loss in an individual.

37. A product according to claim 31, wherein the product is for emulsifier maintaining blood pressure in an individual.

38. A mixture according to claim 25, wherein the triglyceride oil is a confectionary fat.

39. A method according to claim 28, wherein the triglyceride oil is a confectionary fat.
